(19) United States
(12) Patent Application Publication
(10) Pub. No.: US 2008/0124410 A1
(43) Pub. Date: May 29, 2008

(54) Use Of Lavender Oil For The Prophylaxis And Treatment Of Neurasthenia, Somatization Disorders And Other Stress-Associated Diseases

(76) Inventors: Angelika Dienel, Karlsruhe (DE); Michael Noldner, Karlsruhe (DE)

Correspondence Address: EDWARDS ANGELL PALMER & DODGE LLP, P.O. BOX 55874, BOSTON, MA, 02205, US

(21) Appl. No.: 11/664,884

(22) PCT Filed: Oct. 5, 2005
(86) PCT No.: PCT/EP05/10732

§ 371 Date: Jun. 7, 2007

(30) Foreign Application Priority Data
Oct. 6, 2004 (DE) 10 2004 048 716.2

(51) Int. Cl. A61K 36/53; A61P 25/00

(52) U.S. Cl. 424/745

(57) ABSTRACT

The present invention relates to the prophylactic and therapeutic use of lavender oil for the treatment of neurasthenia, somatization disorders and other stress-associated diseases as well as medicaments, dietetic food products, preparations and capsules containing lavender oil as oral administration forms.

The present invention relates to the prophylactic and therapeutic use of lavender oil for the treatment of neurasthenia, somatization disorders and other stress-associated diseases as well as lavender oil containing medicaments and dietetic food products as well as preparations and capsules as oral administration forms.

An acquired nervousness with the symptoms of rapid fatigability, physical weak-ness, headache and pains of the extremities, vegetative hypersensibility, emotional liability, memory deficit and lack of concentration, irritability, mood swings and sleep disorders are referred to as neurasthenia (Roche Lexikon, Medizin, 4^{th} edition 1998).

The presence of multiple, repeatedly occurring and frequently changing physical symptoms are referred to as somatization disorder. The symptoms may relate to each part of the body or each system of the body.

Neurasthenia and somatization disorders may develop because of various causes, whereas stress is frequently an eliciting factor.

Today the physical changes which occur in connection with external undesired stimuli and which contain a variety of physiological reactions which aim at maintaining and stimulating the life-sustaining functions, are usually summarized under the term “stress reaction” (G. A. Carrasco et al. (2003), Eur. J. Pharmacol. 463, 235-272). However, on the one hand the physiological systems which have been activated by stress, have a protective and function-maintaining effect, but on the other hand they may also have harmful results (B. S. McEwen (1998), New Engl. J. Med. 338, 171-179). For example, it is known that stress negatively effects a plurality of diseases and that the chronic influence of stress may also elicit diseases (S. Sephton et al. (2003), Brain Behav. Immun. 17, 321-328; P. H. Black et al. (2002), J. Psychosom. Res. 52, 1-23). In addition to the diseases mentioned above, also diseases of the cardiovascular system, the muscular system and the skeleton as well as disorders of the immune system and the posttraumatic stress disease (PTSD) may be mentioned as examples of these stress-associated diseases.

The currently available medicaments for the treatment of neurasthenia, somatization disorders and other stress-associated diseases comprise anxiolytics such as various benzodiazepines, neuroleptics as well as various antidepressive agents. However, the efficacy of these medicaments are limited. Moreover, they are associated with significant side effects. For example the chronic use of benzodiazepines leads to addiction whereas neuroleptics may elicit very unpleasant, so-called early dyskenisia or tardive dyskenisia. Depending on the medicament used, the use of antidepressive agents frequently leads to vegetative disorders such as xerostomia, tremor, fatigue, or to sexual dysfunctions up to anorgasmy.

Therefore, it is the object underlying the present invention to provide a medicament which may be used effectively in the prophylaxis and/or treatment of neurasthenia, somatization disorders and other stress-associated disorders and which is substantially free of side effects.

This object is solved by the use of lavender oil.

True lavender (Lavendula angustifolia MILL.) grows as a subshrub with a height of about 60 cm. The native range extends from the Canary Islands across the whole Mediterranean region to the Indian peninsula. Particularly essential oils in the aerial parts (primarily monoterpenes) as well as caffeic acid and depsides thereof in the leaves are described as ingredients. The essential oils prepared from lavender are traditionally used for cosmetic products, but also for therapeutic purposes, for example in aromatherapy. For example, antibacterial, antifungal, spasmolytic, sedative and antidepressive effects are describes for lavender oil (H. M. A. Cavanagh et al. (2002), Phytother. Res. 16, 301-308).

In Germany, preparations from lavender flowers in the form of infusions, as an extract as well as a bath additive for the indications conditions of restlessness, disorders in getting to sleep, functional ailment of the abdomen, meteorism and in balneotherapy are positively monographed (monograph of commission E of the former German Federal Health Authority).

It has now been surprisingly discovered that the oral application of lavender oil in patients suffering from neurasthenia, somatization disorders and/or posttraumatic stress disease leads to a significant improvement in various disease-related symptoms. Moreover, stress-induced behavioural changes were significantly inhibited by oral administration of lavender oil in animal experiments. Thus, according to the present invention, the oral uptake of lavender oil can be used for the therapy of neurasthenia, somatization disorders and stress-associated diseases. Such effects have not been described for lavender oil up to now and could not be expected due to the pharmacological and clinical effects which have been heretofore known for lavender oil.

The efficacy of lavender oil was tested in patients suffering from neurasthenia and/or posttraumatic stress disease (PTSD) and/or a somatization disorder. After six weeks of therapy with 80 mg lavender oil per day the core symptoms of neurasthenia, PTSD and somatization disorder were improved. The patient's quality of life showed a considerable improvement (cf. Example 1).

The stress-reducing effects of lavender oil was tested in rats by the so-called “Forced Swimm Stress” model (R. D. Porsolt et al. (1978), Eur. J. Pharmacol. 47, 379-391). The stress reaction experimentally elicited by this test in rats is a recognized method for testing stress-induced behaviour. The principle of the test system used involves that rats being put into a water-filled glass cylinder which they can not leave independently, fall into a still state after a short period of swimming (immobilization period). This immobility is interpreted as a reaction to recognizing the hopelessness of the situation (cf. Example 2).

Lavender oil can be prepared by preparation methods known per se, preferably by steam distillation of freshly harvested lavender flowers.

The lavender oil can be administered in a state of being filled into capsules made of gelatine, cellulose derivatives or other materials suitable for encapsulation or as a solution, preferably orally.

For the preparation of capsules the oil is mixed with suitable pharmaceutically acceptable adjuvants such as mid-chained triglycerides, vegetable oils (e.g. sun-flower oil, soybean oil, wheat germ oil and the like) and filled into capsules. Further adjuvants such as stabilizers are optionally added to the mixture.

The dosage is such that 10 mg to 2 g, preferably 20 to 500 mg and particularly preferred 50 to 100 mg lavender oil are administered per day.

### EXAMPLES

### Example 1

### Efficacy of Lavender Oil in Human Beings

50 patients suffering from neurasthenia and/or posttraumatic stress disease (PTSD) and/or a somatization disorder were treated for six weeks with 80 mg lavender oil per day according to the European Pharmacopoeia, edition 4 (Lavendelöl WS® 1265). The improvement of the pathology was measured and documented using the following recognized test method: Symptom Checklist (SCL 90), State-Trait-Anxiety Inventory (STAI; A. M. Sesti (2000), QoL Newsletter 25, 10-16), depression scale (D-S), Maslach Burnout Inventory (MBI), 36 Item Short Form Survey (SF-36), state check and sleep diary. Furthermore, the patients were hospitalized and surveyed for the symptoms by a physician (third party assessment) or the patients filled out the predetermined questionnaires (self-assessment). Changes in the pathology prior and after the six week therapy were tested using Wilcoxon Signed Rank Test and were statistically significantly improved for the predominant part of the symptoms.

After the six week therapy with 80 mg Lavendelöl WS® 1265 per day, the restless-ness improved in 29 (61.7%) of the patients and the anxiety in 21 (44.7%) of the patients (state check, probability of error p<0.001 for both symptoms; here and in the following: Wilcoxon Signed Rank Test). Both the state anxiety (improvement of 4.5±10.7 points, p=0.005) and the trait anxiety (improvement of 7.4±8.9 points, p<0.001) were alleviated. An improvement of the sleep disorder was shown in 24 (51.5%) of the patients (state check, probability of error p<0.001) and for important items in the sleep diary showed a significant improvement during the treatment phase. The frequency of awakening improved in week 1 from 1.9±0.7 by 0.2±0.6 times (p=0.004), the duration of awakening reduced from 35.6±28.0 by 12.8±24.3 minutes (p<0.001), the total sleep period was extended during the therapy in week 1 from 379.6±59.1 by 16.6±43.0 minutes (p=0.024) and the sleep efficiency was improved in week 1 from 77.4±10.2% by 3.9±8.1% (p=0.001). Moreover, a slight improvement of the morning mood as well as of the fatigue in the morning and a slight increase of the productivity were observed (morning mood: 3.0±0.5 in week 1, improvement by 0.2±0.6 points; fatigue in the morning: 3.4±0.7 in week 1, improvement by 0.3±0.8 points; productivity: 3.0±0.7 in week 1, improvement by 0.2±0.6 points). The depressive mood of 27 (57.4%) patients improved after the six week therapy with Lavendelöl WS® 1265 (state check, p<0.001) and the score on the depression scale (D-S) was lowered by 5.5±7.0 points (p<0.001). All subscores of SCL-90-R and, as a result, the global severity index, positive symptom total and the positive symptom distress index were reduced until week six (improvement of 0.4±0.3 (GSI), 14±12.4 (PST) and 0.4±0.4 (PSDI) points, p<0.001 for all the three global scores. The physical health score and the mental health score of SF-36 significantly increased after six week treatment by 9.8±15.7 and by 20.8±22.3 points (p<0.001 for both scores), respectively. Thus, the results of the study demonstrate a consider-able improvement of the pathology in case of neurasthenia, somatization disorder and other stress-related diseases by taking lavender oil.

### Example 2

### Efficacy of Lavender Oil in Rats

In order to test the stress-reducing effects of lavender oil, rats were treated over a period of nine days in total with differently high dosages of lavender oil according to European Pharmacopoeia, edition 4 (WS® 1265) once a day. For comparison purposes some animals were treated either with the tricyclic antidepressive agent imipramine used in stress therapy or with a control solution without an active ingredient. On the seventh day of the treatment, the animals were placed into the water-filled glass cylinder for 15 minutes for familiarization purposes. At the final day of the treatment, the animals were placed into the graduated cylinder again for a period of 5 minutes and the period of immobility was measured as a measure of the stress reaction. Oral application of lavender oil over a period of nine days at a dosage of 10 to 100 mg/kg led to a considerable, significant reduction of the immobilization period.

| | dosis | immobilization period | inhibition |
|---|---|---|---|
| substance | mg/kg perorally | seconds | % |
| control | | 146 ± 11 | 0 |
| lavender oil | 1 | 149 ± 8 | 0 |
| lavender oil | 3 | 136 ± 12 | 7 |
| lavender oil | 10 | 116 ± 13 * | 21 |
| lavender oil | 30 | 83 ± 5 * | 43 |
| lavender oil | 100 | 80 ± 17 * | 45 |
| imipramine | 30 | 48 ± 6 * | 67 |

* probability of error p < 0.05 versus control

### Example 3

### Capsules

An amount of 800 g lavender oil which is required for the preparation of about 10,000 capsules, is mixed with 1200 g mid-chained triglycerides in a tightly sealed inert container (for example an agitator vessel made of stainless steel) and mixed for 15 minutes. The homogenous liquid mixture is filled into gelatine capsules in an amount of 200 mg per capsule using a suitable capsule filling apparatus. In the case of hard capsules (for example made of gelatine or cellulose derivatives), the capsules are sealed by means of a sleeve after filling. In case of capsules made of soft gelatine, the capsules are filled and sealed in one operation.

1-4. (canceled)

5. A method for treating a subject suffering from or susceptible to neurasthenia, a somatization order or posttraumatic stress disease, comprise orally administering lavender oil to the subject.

6. The method of claim 5 wherein a capsule comprising lavender oil is administered.

7. The method of claim 5 wherein the subject is identified as suffering from or susceptible to neurasthenia, a somatization order or posttraumatic stress disease and the lavender oil is administered to the identified subject.

8. The method of claim 5 wherein the subject is identified as suffering from neurasthenia, a somatization order or posttraumatic stress disease and the lavender oil is administered to the identified subject to thereby treat the patient for neurasthenia, a somatization order or posttraumatic stress disease.

9. A medicament or dietetic food product comprising lavender oil.

10. The medicament or dietetic food product of claim 9 packaged together with instructions for use of the medicament or dietetic food product for the treatment or prophylaxis of neurasthenia, somatization order or posttraumatic stress disease.

11. A preparation comprising lavender oil and one or more pharmaceutically acceptable adjuvants in an oral administration form.

12. The preparation of claim 11 packaged together with instructions for use of the preparation for the treatment or prophylaxis of neurasthenia, somatization order or posttraumatic stress disease.

13. The preparation of claim 11 wherein the preparation consists essentially of lavender oil and one or more pharmaceutically acceptable adjuvants in an oral administration form.

14. The preparation of claim 12 wherein the preparation consists essentially of lavender oil and one or more pharmaceutically acceptable adjuvants in an oral administration form.

15. The preparation of claim 11 wherein the preparation consists of lavender oil and one or more pharmaceutically acceptable adjuvants in an oral administration form.

16. The preparation of claim 12 wherein the preparation consists of lavender oil and one or more pharmaceutically acceptable adjuvants in an oral administration form.
